(19) United States
(12) Patent Application Publication
(10) Pub. No.: US 2024/0185946 A1
(43) Pub. Date: Jun. 6, 2024

(54) METHOD FOR IDENTIFYING A CHROMATIN STRUCTURAL CHARACTERISTIC FROM A HI-C MATRIX, NON-TRANSITORY COMPUTER READABLE MEDIUM STORING A PROGRAM FOR IDENTIFYING A CHROMATIN STRUCTURAL CHARACTERISTIC FROM A HI-C MATRIX, AND METHODS FOR DIAGNOSING AND TREATING A MEDICAL CONDITION OR DISEASE

(71) Applicant: CHROMATINTECH BEIJING CO, LTD, Beijing (CN)

(72) Inventors: Jingyao WANG, Beijing (CN); Yue XUE, Beijing (CN); Yueying HE, Beijing (CN); Yiqin GAO, Beijing (CN)

(73) Assignee: CHROMATINTECH BEIJING CO, LTD, Beijing (CN)

(21) Appl. No.: 17/796,425

(22) PCT Filed: Nov. 23, 2021
(86) PCT No.: PCT/CN2021/132568

§ 371 Date: Jul. 29, 2022

(51) Int. Cl. G16B 15/00; G16H 50/20

(52) U.S. Cl. CPC G16B 15/00; G16H 50/20

(57) ABSTRACT

A method for identifying a chromatin structural characteristic from a Hi-C matrix, a non-transitory computer readable medium storing a program for identifying a chromatin structural characteristic from a Hi-C matrix, and methods for diagnosing and treating a medical condition or disease. The method for identifying a chromatin structural characteristic from a Hi-C matrix includes performing a correlation process on the Hi-C matrix to calculate a correlation matrix, calculating a structural characteristic vector based on the correlation matrix, calculating a principal component fraction matrix from the structural characteristic vector, and identifying at least one chromatin structural characteristic in the principal component fraction matrix.

### TECHNICAL FIELD

Embodiments of this application relates to a method for identifying a chromatin structural characteristic from a Hi-C matrix, a non-transitory computer readable medium storing a program for identifying a chromatin structural characteristic from a Hi-C matrix, and methods for diagnosing and treating a medical condition or disease such as cancer.

### BACKGROUND

Chromosome conformation capture techniques (often abbreviated to 3 C technologies or 3C-based methods) are a set of molecular biology methods used to analyze the spatial organization of chromatin in a cell. These methods quantify the number of interactions between genomic loci that are nearby in 3D space, but may be separated by many nucleotides in the linear genome. Interaction frequencies may be analyzed directly, or they may be converted to distances and used to reconstruct 3D structures.

High-throughput chromosome conformation capture (Hi-C) allows for genome-wide profiling of chromatin interactions in space and has been used to study the genome-wide interactions of genomes. It is well known that spatial organization of chromatin is non-random and is crucial for deciphering how the 3D architecture of DNA affects genome functionality and transcription. Hi-C technology provides a deeper insight into the 3D organization of chromatin by comprehensive detection of spatial interactions between genomic regions. Hi-C technology typically involves the production of hundreds of millions of paired-end sequencing reads. It can capture chromatin interactions across an entire genome and construct a genome-wide Hi-C contact matrix, where each element in the matrix denotes the contact strength between any two regions of genome.

Hi-C uses high-throughput sequencing to find the nucleotide sequence of fragments and uses paired end sequencing, which retrieves a short sequence from each end of each ligated fragment. As such, for a given ligated fragment, the two sequences obtained should represent two different restriction fragments that were ligated together in the proximity based ligation step. The pair of sequences is individually aligned to the genome, thus determining the fragments involved in that ligation event. Hence, all possible pairwise interactions between fragments are tested.

Presently, Hi-C is the most commonly used method to detect chromatin structure. The pairing data generated by this technology is processed by existing pipelines. The generated data format is a Hi-C matrix, which is a spatial contact frequency matrix between chromatin sites. There are currently are many mathematical methods available to model chromatin conformation from a Hi-C matrix, including generating spatial distance matrix from Hi-C matrix and/or predicting the coordinates of chromatin sites in 3D space.

Hi-C is used to calculate multi-scale chromatin structural features to characterize cell state. Existing solutions include (1) decomposing the Hi-C matrix into one-dimensional vectors by non-negative matrix decomposition, which can be used to characterize the topological domain of chromatin, (2) principal component analysis (PCA) of a Hi-C matrix, in which the physical meaning of the first principal component is the degree of a DNA bin to interior of two heterogeneous regions (compartment A/B), (3) starting from the Hi-C matrix, performing Monte Carlo simulation to obtain the simulated physical conformation of chromatin, and then extracting the structural features, and (4) using chromatin accessibility data (DNase, MNase) to characterize the structural characteristics of chromatin.

The shortcoming of existing methods for detecting chromatin transcription initiation sites, such as chromatin accessibility data, is that they represent the chromatin structural features on a relatively small scale (like nucleosome scale), and fail to provide structural features in multiple distance scales. The disadvantage of first principal component (PC1), i.e., partition of two arms of chromatin, from PCA of a Hi-C matrix to characterize the structure is that for Hi-C matrix with poor data quality (low sequencing depth and high noise), the result is not reliable. The disadvantage of performing Monte Carlo simulation from a Hi-C matrix to obtain the 3D spatial conformation and the structural features is that additional assumptions are needed, such as the relationship between contact frequency and physical distance. These assumptions do not necessarily conform to reality, so deviations exist in calculated conformation and features. The disadvantage of non-negative matrix decomposition based on a Hi-C matrix to characterize the structure is that it only characterizes the boundary of topology associated domains (TADs), rather than the structural features inside TADs.

Accurately characterizing the structural characteristics of chromatin sites along the sequence is important for diagnosis and treatment of medical conditions or disease with a genetic basis such as cancer. By looking for specific chromatin interactions that exist only in cancer or only in normal cells, potential locus associated with cancer can be identified. Therefore, there is a significant need in bioinformatics for methods that are useful in identifying chromatin structure and differences between structures in normal versus aberrant cells. These and other problems are addressed by the following disclosed embodiments.

### SUMMARY

The inventors found that chromatin structural characteristics along the DNA sequence can be used to characterize the cell type, and that the chromatin structural differences between normal cells and aberrant cells along the sequence can be used to distinguish normal cells and aberrant cells.

In particular, the inventors found that by calculating structural characteristic vectors along sequences in input Hi-C matrix datasets and identifying normal cells and aberrant cells using a cell type atlas it is possible to more reliably and more efficiently find the difference in chromatin structure between different types of cells. They also found that such methods could be very useful in diagnosing and treating a myriad of medical conditions or diseases including, but not limited to, cancer. According to disclosed embodiments, identifying chromatin structural characteristics from a Hi-C matrix is possible in a novel and surprisingly effective manner.

In a first embodiment, there is provided a method for identifying a chromatin structural characteristic from a Hi-C matrix. The method includes performing a correlation process on the Hi-C matrix to calculate a correlation matrix, calculating a structural characteristic vector based on the correlation matrix, calculating a principal component fraction matrix from the structural characteristic vector, and identifying at least one chromatin structural characteristic in the principal component fraction matrix.

In another embodiment, there is provided a non-transitory computer readable medium storing a program for identifying a chromatin structural characteristic from a Hi-C matrix. The program causes the processor to execute performing a correlation process on the Hi-C matrix to calculate a correlation matrix, calculating a structural characteristic vector based on the correlation matrix, calculating a principal component fraction matrix from the structural characteristic vector, and identifying at least one chromatin structural characteristic in the principal component fraction matrix.

In another embodiment, there is provided a method for diagnosing a medical condition or disease. The method includes identifying the chromatin structural characteristic according to disclosed methods, and relating the chromatin structural characteristic to a medical condition or disease.

In another embodiment, there is provided a method for treating a medical condition or disease. The method includes identifying the chromatin structural characteristic according to disclosed methods, and administering a gene therapy vector to a subject in need thereof. The chromatin structural characteristic is indicative of a medical condition or disease.

### BRIEF DESCRIPTION OF THE DRAWINGS

To describe the technical solutions in embodiments of the present invention or in the prior art more clearly, the following briefly introduces the accompanying drawings needed for describing the embodiments or the prior art. Apparently, the accompanying drawings in the following description illustrate merely some embodiments of the present invention, and persons of ordinary skill in the art may still derive other drawings from these accompanying drawings without creative effort.

FIG. 1 is a schematic illustration of a method for identifying structural characteristics from a Hi-C matrix according to embodiments.

FIG. 2 is a schematic illustration of sub-steps of a method for generating an enhanced Hi-C matrix according to an embodiment.

FIG. 3 is a schematic illustration of algorithm flow for obtaining a structural eigenvector from a Hi-C matrix according to an embodiment.

FIG. 4 is a schematic illustration of the algorithm flow of principal component analysis using structural eigenvectors according to an embodiment.

FIG. 5(a) and FIG. 5(b) illustrate Hi-C matrix heatmaps for normal cells (FIG. 5(a)) and cancer cells (FIG. 5(b)) according to an embodiment.

FIG. 5(c) and FIG. 5(d) illustrate correlation matrix heatmaps for normal cells (FIG. 5(c)) and cancer cells (FIG. 5(d)) according to an embodiment.

FIG. 5(e) and FIG. 5(f) illustrate converted logical matrix heatmaps for normal cells (FIG. 5(e)) and cancer cells (FIG. 5(f)) according to an embodiment.

FIG. 6 illustrates a structural eigenvectors plot according to an example of an embodiment.

FIG. 7 illustrates a PCA-derived cell type atlas according to an example of an embodiment.

FIG. 8 illustrates a PCA-derived cell type atlas according to an example of an embodiment.

FIG. 9 illustrates a PCA-derived cell type atlas according to an example of an embodiment.

### DESCRIPTION OF EMBODIMENTS

To make the objectives, technical solutions, and advantages of embodiments of the present invention clearer, the following clearly and comprehensively describes the technical solutions in embodiments of the present invention with reference to the accompanying drawings in embodiments of the present invention. Apparently, the described embodiments are merely a part rather than all embodiments of the present invention. All other embodiments obtained by persons of ordinary skill in the art based on embodiments of the present invention without creative effort shall fall within the protection scope of the present invention.

Disclosed methods start from an input Hi-C matrix. A correlation matrix is calculated from the Hi-C matrix and represents a description of similarity between chromatin bins. Based on the correlation matrix, structural characteristic vectors are calculated. Each element of the structural characteristic vector is the structural feature of each bin along the DNA sequence. Structural characteristic vectors under multiple scales can be used to characterize the cell state.

The structural characteristic vectors of different cell types are different. For example, the structural vectors of normal cells and aberrant cells, such as cancer cells, are clearly distinguishable. The positions of vector elements with the most significant difference between normal cells and cancer cells are potential targets of cancer detection and anti-cancer therapy. Specifically, positions of those elements are locations of chromatin bins undergoing significant structural changes during tumorigenesis. By taking those chromatin bins as targets, early screening of cancer and discrimination of cancer stages can be realized. The chromatin bins may also serve as targets of anti-cancer therapy.

Methods for identifying a chromatin structural characteristic from a Hi-C matrix

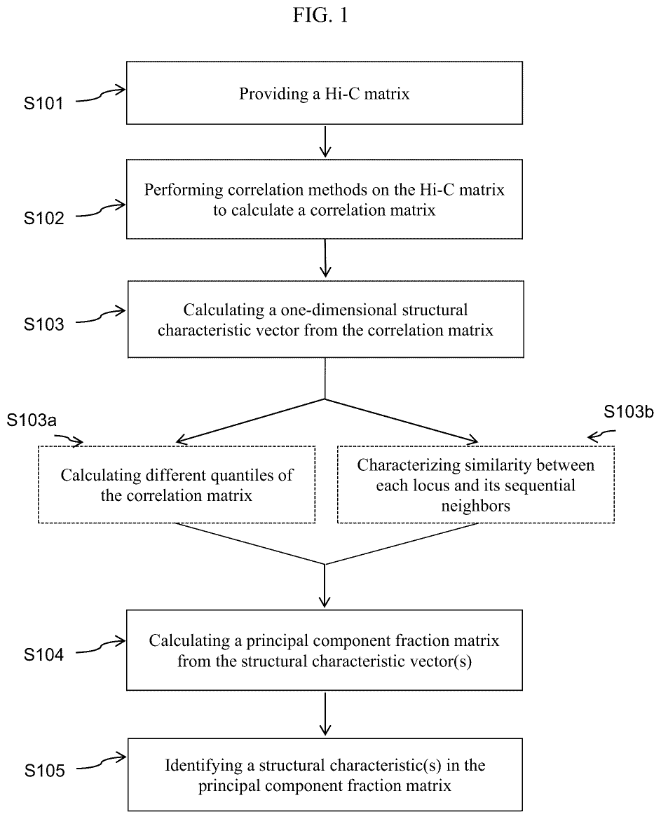

With reference to FIG. 1, disclosed methods may start from an input Hi-C matrix in step S101. Hi-C matrices are well-known in the art. The Hi-C matrix may be a raw-data, normalized, or enhanced Hi-C matrix. The input may also include a single cell Hi-C matrix or other similar sequencing technologies aimed at chromatin conformation capture.

In step S102, correlation methods are applied to the Hi-C matrix to calculate or derive a correlation matrix through multiple iterations to remove noise and underline higher order correlation between chromatin bins. Correlation methods according to embodiments are not particularly limited and may include, but are not limited to, Pearson correlation, Spearman correlation, and cosine similarity. The number of iterations employed in embodiments is also not particularly limited and may be, for example, in the range of 1 to 20, 1 to 10, or 1 to 4.

The derived correlation matrix from step S102 may be used as a measure of the similarity between chromatin bins by deriving a structural characteristic vector in step S103. The structural characteristic vector may be, for example, a one-dimensional structural characteristic vector. In embodiments, the structural characteristic vector may be derived by calculating different quantiles of the correlation matrix in step S103a and/or by characterizing similarity between each locus and its sequential neighbors in step S103b.

In embodiments, the structural characteristic vector may be derived by calculating different quantiles of the correlation matrix, as in step S103a. In step S103a, different quantiles of the correlation matrix may be calculated and taken as a threshold of transforming the correlation matrix to a binary matrix. For example, for each quantile, the correlation matrix may be converted into a logical matrix consisting of 0 and 1 specifically, and matrix elements greater than the quantile and less than the quantile are converted to 1 and 0, respectively, or vice versa. A one-dimensional vector is obtained by summing the rows of the logical matrix. The one-dimensional vector is a structural characteristic under the scale defined by the quantile, and its length is the number of chromatin bins. The structural characteristics under different scales are calculated from different quantiles. The number of quantiles used represents the scale range taken into account. In other embodiments, step S103a may be implemented by using different specific values instead of different quantiles, as will be appreciated by one of ordinary skill in the art.

In embodiments, the structural characteristic vector may be derived by characterizing similarity between each locus and its sequential neighbors, as in step S103b. In this step, for each locus, the average similarity between each 2 loci on a window centered around the locus is calculated. For a designated window size and a locus, a sub-matrix is extracted from the correlation matrix. Next, for the window size and the locus, the sub-matrix is averaged into a one-dimensional vector with a length equal to the number of chromatin bins. The window sizes may be chosen consecutively and may encompass any suitable range of loci. For example, the window may be in the range of 1 to 10,000, 1 to 5,000, 1, 1,000, 2 to 500, 3 to 100, or 5 to 50 loci. It will be understood that different window sizes represent different scales taken into consideration and that window size may be dependent upon a variety of factors including, but not limited to, the target medical condition or disease and the corresponding genomic profile(s).

The one-dimensional vector at different scales obtained in step S103 is considered the structural characteristic vector. After calculating the structural characteristic vector of each sample in step S103, the next step is calculating a principal component fraction matrix from the structural characteristic vector, as in step S104.

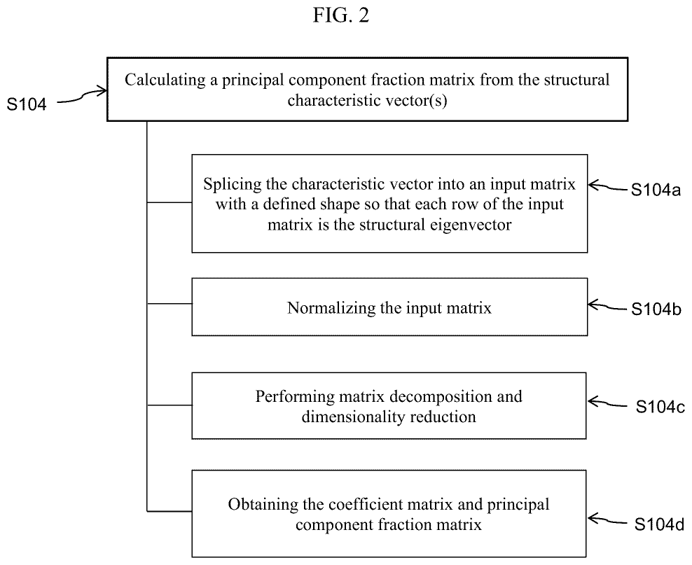

With reference to FIG. 2, calculating the principal component fraction matrix from the structural characteristic vector may include, in step S104a, for each sample having a characteristic vector with a defined shape, splicing the characteristic vector into an input matrix with a defined shape so that each row of the input matrix is the structural eigenvector of a sample, and each column corresponds to a chromatin bin along the DNA sequence.

Calculating the principal component fraction matrix from the structural characteristic vector may include, in step S104b, normalizing the input matrix, and in step S104c, performing matrix decomposition and dimensionality reduction. Any suitable matrix decomposition and dimensionality reduction methods may be performed. In embodiments, the matrix decomposition and dimensionality reduction method may include, but is not limited to, PCA, non-negative matrix decomposition and eigenvalue decomposition, and singular value decomposition (SVD) algorithm. In this step, rows of the input matrix correspond to observations and columns correspond to variables. The initial values of coefficient matrix and principal component fraction matrix are random matrices. The number of iteration steps may be as high as 1,000 and may be in a range of 2 to 1,000, 5 to 500, 10 to 200, or 20 to 100. The tolerance of the loss function may be up to 1e-6.

In step S104d, the coefficient matrix and principal component fraction matrix are obtained from step S104a. In embodiments, each column of the coefficient matrix contains coefficients for one principal component, and the columns are in descending order of component variance. The principal component fraction matrix is the representation of the input matrix in the principal component space. The rows of the principal component fraction matrix correspond to the samples and the columns correspond to the principal components.

In step S105, structural characteristic(s) in the principal component fraction are identified. In this step, taking the obtained principal component fraction matrix as the representation of samples, geometric visualization approaches may be performed to identify the relationship between samples from step S104. Any suitable geometric approach may be performed. In embodiments, the geometric approach may include, but is not limited to, embedding the principal components in a visualized cell type atlas.

The disclosed methods for identifying a chromatin structural characteristic from a Hi-C matrix will now be described with respect to the following sample algorithms for further understanding of the disclosed embodiments. However, the disclosure is not intended to be limited to the specific algorithms described below.

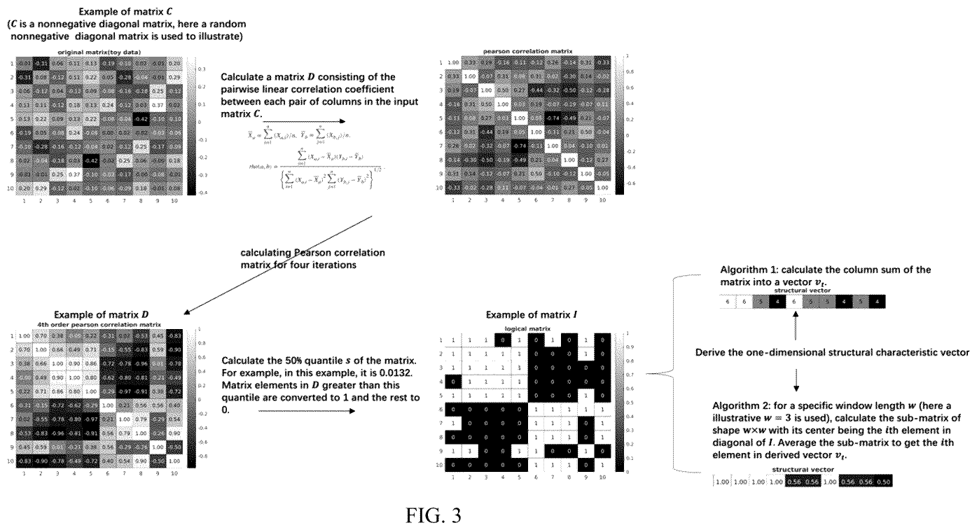

In embodiments, the following operations are exemplified using the normalized random non-negative diagonal matrix C in FIG. 3. To the input matrix C, a Pearson correlation is performed to calculate the correlation matrix of C, i.e., correlation matrix D, for four iterations to remove noise and underline higher order correlation between chromatin bins. In this case, the correlation matrix D consists of the pairwise linear correlation coefficient between each pair of columns in the input matrix C. The derived correlation matrix D is used as a measure of the similarity between chromatin bins. The correlation matrix D is used as input of Algorithms I and II described below.

In Algorithm I, the different quantiles of the correlation matrix D are calculated. For each quantile s, the correlation matrix D is converted into a logical matrix Is consisting of 0 and 1 specifically, and any matrix element greater than s and less than s is converted to 1 and 0, respectively. In this case, the 50% quantile s of the correlation matrix D is calculated, i.e., 0.0132, as seen in FIG. 3. Elements in correlation matrix D greater than this quantile are converted to 1 and the rest to 0. The one-dimensional vector ve is obtained by summing the row of logical matrix IS. The one-dimensional vector ve is a structural characteristic under the scale defined by the quantile s, and its length is the number of chromatin bins N. The structural characteristics under different scales are calculated from different quantiles. The number of quantiles used represents the scale range taken into account. The derived vector vₜ results.

In Algorithm II, the similarity between each locus and its sequential neighbors is characterized. Specifically, for each locus i, the average similarity between each 2 loci on a window centered around i calculated in the following steps:
i) For a certain window size w and a locus i, the sub-matrix Dᵢ^{w} from the correlation matrix D is extracted. Dᵢ^{w} consists of d_{ab} (i−w≤a≤i+w, i−w≤b≤i+w), where d_{ab} is the element at row a and column b of D.
ii) For a certain window size w and a locus i, the sub-matrix DW is averaged into a value vₜ^{w}(i) and the one-dimensional vector vₜ^{w} is derived with a length equal to the number of chromatin bins N.

In this case, the specific window length w is 3 and a sub-matrix was calculated with the shape w×w with its center being the ith element in diagonal matrix I. The sub-matrix was averaged to get the ith element in derived vector vₜ.

The one-dimensional vector vₜ at different scales obtained by Algorithms I and II is defined as the structural characteristic vector. After calculating vₜ of each sample, PCA was carried out according to Algorithm III.

In Algorithm III, n samples were taken. Each of them has characteristic vector vₜ with shape 1×N. All vₜ are spliced vertically into a matrix X with shape n×N so that each row of input X is the structural eigenvector vₜ of a sample, and each column corresponds to a chromatin bin along the DNA sequence. Then, the following steps were applied:
i) X was normalized.
ii) PCA was carried out on X. Rows of X correspond to observations and columns correspond to variables. The SVD algorithm was applied. The initial values of coefficient matrix coeff and principal component fraction matrix score are random matrices. The maximum number of iteration steps allowed was 1000. The tolerance of the loss function was 1e-6.
iii) Coefficient matrix coeff and score were obtained. The coefficient matrix coeff is N×N and the principal component fraction matrix score is n×p, where p is number of principle components. Each column of coeff contains coefficients for one principal component, and the columns are in descending order of component variance. Principal component fraction matrix score is the representation of X in the principal component space. The rows of score correspond to the samples and the columns correspond to the principal components.

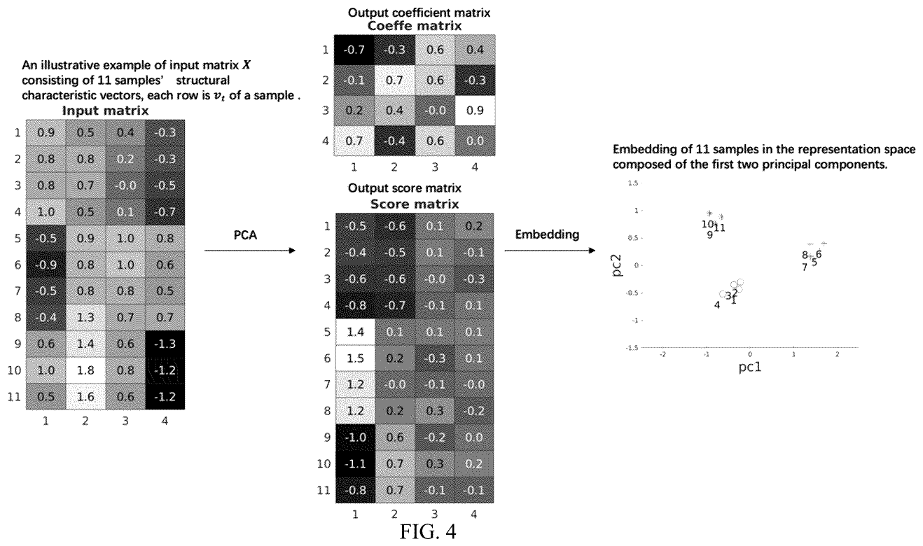

Geometric approaches were then be used to find the relationship between samples taking the obtained score as the representation of samples. FIG. 4 illustrates the algorithm flow of principal component analysis using structural eigenvectors. In FIG. 4, each row of the input matrix correspond to the structural eigenvectors obtained above. Structural eigenvectors of 11 samples were calculated here, the length of each vector was 4, and the input matrix X was 11× 4, i.e., chromatin bins N=4. It will be appreciated that the number of chromatin bins N is not particularly limited and may be, for example, in a range of 100 to 20,000, 500 to 10,000, or 1000 to 6,000.

Each element of the structural characteristic vector vₜ is the structural feature of each bin along the DNA sequence. In this case, cancer cells refer to a variety of solid tumor cancers, leukemia, as well as a variety of cancer cell lines and leukemia cell lines, and normal cells refer to corresponding normal cells of specific cancer. The clustering of the structural characteristic vector vₜ in FIG. 4 is very evident.

### Methods for Diagnosing and Treating a Medical Condition or Disease

In other embodiments, there are provided methods for diagnosing and treating a medical condition or disease. The methods include identifying the chromatin structural characteristic in the visualized cell type atlas described above. The obtained visualized cell type atlas also allows for distinguishing normal and cancer cells, describing the development of cancer, and distinguishing different cancers, which are useful in targeting and treatment of a medical condition or disease, such as cancer. Treatment may involve the usage of transcription or translation production of the obtained loci as a medical condition or disease target. This step may include identifying at least one locus associated with the structural chromatin aberration in the target cells. The at least one locus may include, but is not limited to, SPAG9, TOB1, and UTP18.

In the method of diagnosing a disease, the the chromatin structural characteristic is indicative of a disease. In the method of treating a disease, the method includes administering a gene therapy vector to a subject in need thereof. The gene therapy may include usage of transcription or translation production of at least one locus associated with the chromatin structural characteristic in the target cells as a disease target.

According to the disclosed methods, it is possible to identify regulatory genes or regulatory elements capable of modulating open reading frame sequences through physical interactions (close spatial proximity) between these regulatory elements and these open reading frames. The regulatory elements and open reading frame can be located near or far apart along the linear genome sequence or can be located on different chromosomes. The open reading frame sequences may be associated with a medical condition or disease.

In particular, it is possible to find the loci that are prone to change in medical condition or disease such as, for example, cancer, as the target of disease diagnosis and treatment. The inventors found that different types of cancer samples show highly consistent characteristics, indicating that this method is surprisingly effective in identifying the common characteristics of cancer cell structure, and providing new ideas for cancer diagnosis and treatment.

Disclosed embodiments are applicable to and operable on any medical condition or disease with a genetic basis. In this regard, the medical condition or disease may include, but is not limited to, cancer, cardiovascular disease, kidney disease, autoimmune disease, pulmonary disease, liver disease, lymphoid disease, bone marrow disease, bone disease, blood disorder, and the like.

### Non-Transitory Computer Readable Medium and Machine Learning

Disclosed embodiments further include a non-transitory computer readable medium storing a program for identifying a chromatin structural characteristic from a Hi-C matrix, the program causing the processor to execute the disclosed methods. Disclosed embodiments may further include a variety of machine learning algorithms implemented on specialized computers or computer systems for executing any one or more of the disclosed methods. In this regard, the algorithms may be used for automatically executing steps using commercial or open source tools. Machine learning algorithms may be used for mathematically processing large genomic datasets and may also be used in optimizing calculations and increasing the precision and accuracy of outputs.

As is understood in the art of bioinformatics, machine learning algorithms involve establishing classifiers and training datasets. Classifiers play an important role in the analysis of complex multi-dimensional systems, such as chromatin structures and eukaryotic genomes. To develop classifications, supervised learning technology may be based on decision trees, on logical rules, or on other mathematical techniques such as linear discriminant methods (including perceptrons, support vector machines, and related variants), nearest neighbor methods, Bayesian inference, neural networks, and the like.

The programmatic tools used in developing the disclosed machine learning algorithms are not particularly limited and may include, but are not limited to, open source tools, rule engines such as Hadoop®, programming languages including SAS®, SQL, R, Matlab, and Python and various relational database architectures. In embodiments, Python is the preferred programming construct within which to execute disclosed methods.

The specialized computer or processing system that may implement disclosed methods and machine learning algorithms may be a specialized processing system and may be operational with numerous other general purpose or special purpose computing system environments or configurations, as would be understood by a bioinformatics practitioner. Examples of well-known computing systems, environments, and/or configurations that may be suitable for use with disclosed methods may include, but are not limited to, personal computer systems, server computer systems, thin clients, thick clients, handheld or laptop devices, multiprocessor systems, microprocessor-based systems, set top boxes, programmable consumer electronics, network PCs, minicomputer systems, mainframe computer systems, and distributed cloud computing environments that include any of the above systems or devices, and the like.

The computer system may be described in the general context of computer system executable instructions, such as program modules, being executed by a computer system. Generally, program modules may include routines, programs, objects, components, logic, data structures, and so on that perform particular tasks or implement particular abstract data types. The computer system may be practiced in distributed cloud computing environments where tasks are performed by remote processing devices that are linked through a communications network. In a distributed cloud computing environment, program modules may be located in both local and remote computer system storage media including memory storage devices.

Neural networks may be employed in executing disclosed methods. The neural network may be a deep convolutional neural network. The neural network may be a deep neural network that comprises an output layer and one or more hidden layers. In embodiments, training the neural network may include training the output layer by minimizing a loss function given the optimal set of assignments, and training the hidden layers through a backpropagation algorithm.

The deep neural network may be a Convolutional Neural Network (CNN). In a CNN-based model, a set of filters are used to extract features using convolution operation. Training of the CNN is done using a training dataset, which determines the trained values of the parameters/weights of the neural network.

In some CNN models, the numbers of the CNN layers and fully connected layers may vary. In some network architectures, residual pass or feedbacks may be used to avoid a conventional problem of gradient vanishing in training the network weights. The network may be built using any suitable computer language such as, for example, Python or C++. Deep learning toolboxes such as TensorFlow, Caffe, Keras, Torch, Theano, CoreML, and the like, may be used in implementing the network. These toolboxes are used for training the weights and parameters of the network. In some embodiments, custom-made implementation of CNN and deep learning algorithms on special computers with Graphical Processing Units (GPUs) are used for training, inference, or both. The inference is referred to as the stage in which a trained model is used to infer/predict the testing samples. The weights of a trained model are stored in a computer disk and then used for inference. Different optimizers such as the Adam optimization algorithm, and gradient descent may be used for training the weights and parameters of the networks. In training the networks, hyperparameters may be tuned to achieve higher recognition and detection accuracies. In the training phase, the network may be exposed to the training data through several epochs. An epoch is defined as an entire dataset being passed only once both forward and backward through the neural network.

The network can be trained using a transfer learning mechanism. In transfer learning, the network's weights are initially trained using a datatset different than the target dataset to learn the relevant features. Then, this pre-trained network is retrained further using the features in the target database. The CNN architecture can be 3D to handle 3D chromatin structural data.

### EXAMPLES

### Example 1

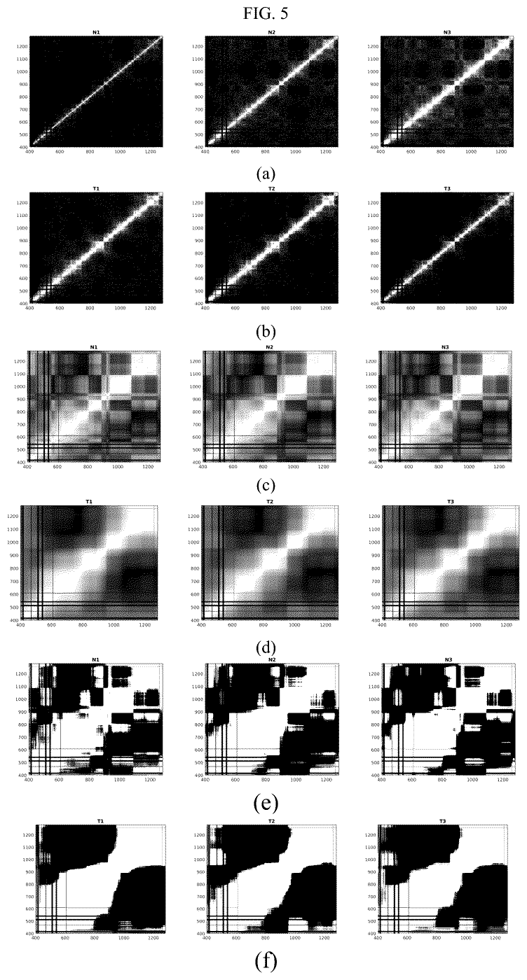

A correlation matrix from an Hi-C matrix was calculated to distinguish between three normal and three cancer oral samples, as seen in FIGS. 5 (a)-(f). First, an original untreated Hi-C matrix heatmap for normal cells (FIG. 5(a)) and cancer cells (FIG. 5(b)) was generated. As seen in FIGS. 5(a) and (b), the distinction between normal and tumor samples is ambiguous.

FIGS. 5 (c) and (d), corresponding to FIGS. (a) and (b), respectively, illustrate heatmaps after Pearson correlation matrix of the fourth iteration was applied. As seen in FIGS. (c) and (d), the distinction between normal and cancel samples is prominent in the calculated correlations matrices.

FIGS. (e) and (f), corresponding to FIGS. (c) and (d), respectively, illustrate heatmaps of converted logical matrices (composed of 0 and 1) obtained by taking 50% quantile of the previous correlation matrices as a threshold. As seen in FIGS. (e) and (f), the distinction between normal and cancel samples is prominent in the calculated logical matrices.

### Example 2

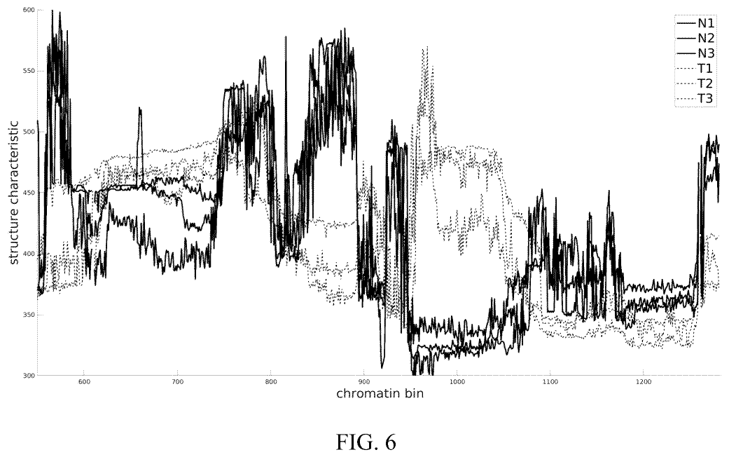

One-dimensional structural eigenvectors vₜ plots were generated for each of three normal oral samples (i.e., N1, N2, and N3) and three cancer oral samples (i.e., T1, T2, and T3) on chromosome 22. The results are illustrated in FIG. 6. As seen in FIG. 6, the cancer samples are easily distinguishable from the normal samples.

### Example 3

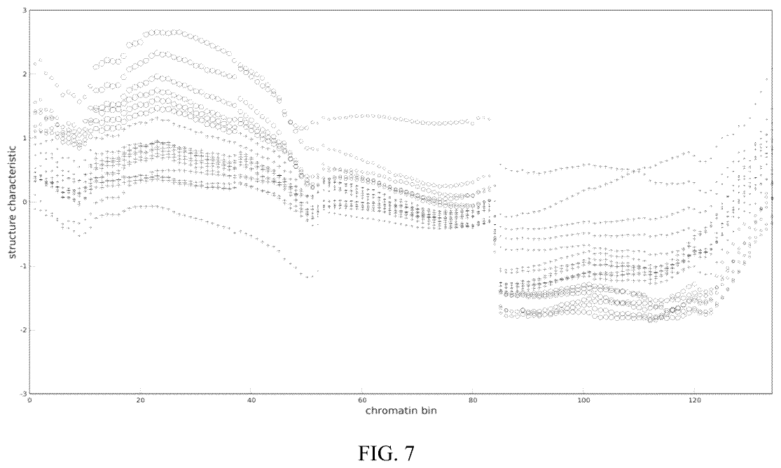

PCA was carried out for one-dimensional structural eigenvectors vₜ of 19 samples to generate a PCA-derived cell type atlas. The results are illustrated in FIG. 7. The first two dimensions of obtained principal components were used to represent samples. In FIG. 7, each line represents the vₜ value of a sample. Lines consisting with + and o represent cancer and normal samples' vₜ, respectively. Structural eigenvectors vₜ values of the 19 samples involving three cancers (oral, colon and bladder) are shown. The resultant cell type atlas in FIG. 7 clearly distinguishes between normal and cancer samples, and samples of different cancers.

### Example 4

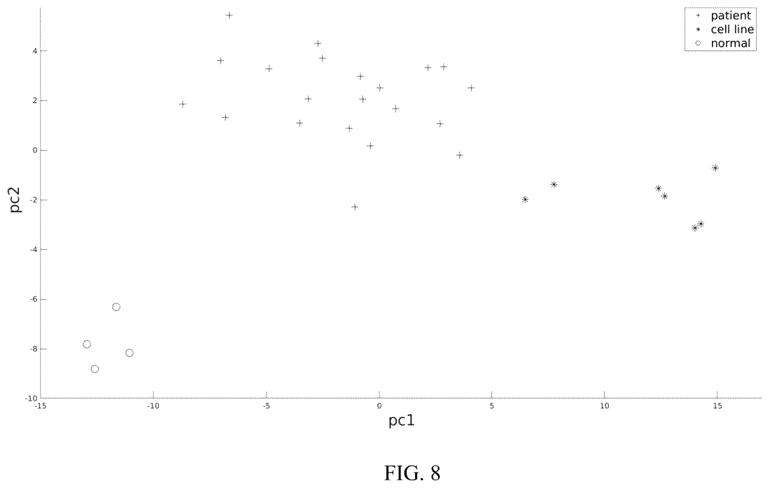

PCA was carried out for one-dimensional structural eigenvectors vₜ of 33 samples to generate a PCA-derived cell type atlas. The results are illustrated in FIG. 8. The first two dimensions of obtained principal components were used to represent samples. In the 33 samples, there were 4 normal blood samples (represented by “o”), 22 leukemia samples (represented by “*”), and 7 leukemia cell line samples (represented by “+”). As seen in FIG. 8, normal blood, leukemia and leukemia cell line samples are clearly shown as 3 clusters and each cell type is distinguishable from the other types.

### Example 5

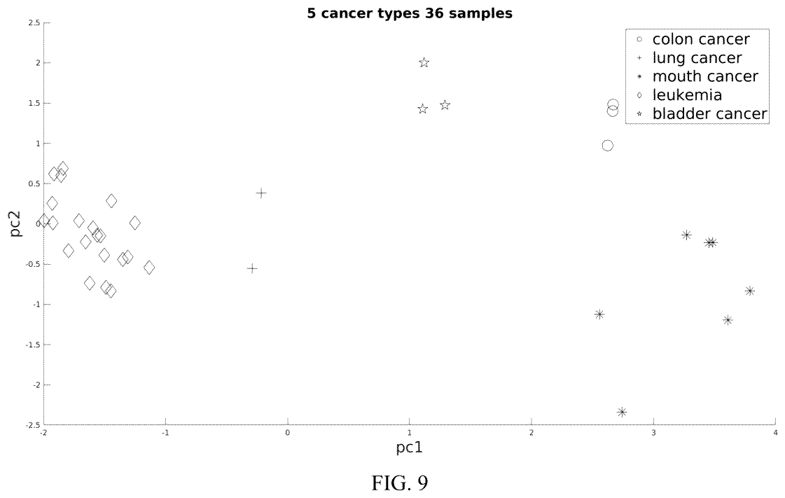

PCA was carried out for one-dimensional structural eigenvectors vₜ of 36 cancer samples to generate a PCA-derived cell type atlas. The results are illustrated in FIG. 9. The first two dimensions of obtained principal components were used to represent samples. In the 36 cancer samples, there were 21 leukemia samples (represented by diamonds), 7 mouth cancer samples (represented by “*”), 3 colon cancer samples (represented by “o”), 3 bladder cancer samples (represented by pentagrams) and 2 lung cancer samples (represented by “+”). As seen in FIG. 9, the 5 cancer types are clearly shown as 5 clusters and each cell type is distinguishable from the other types.

It will be appreciated that the above-disclosed features and functions, or alternatives thereof, may be desirably combined into different devices, systems, and methods. Also, various alternatives, modifications, variations or improvements may be subsequently made by those skilled in the art, and are also intended to be encompassed by the disclosed embodiments. As such, various changes may be made without departing from the spirit and scope of this disclosure.

What is claimed is:

1. A method for identifying a chromatin structural characteristic from a Hi-C matrix, the method comprising:
performing a correlation process on the Hi-C matrix to calculate a correlation matrix;
calculating a structural characteristic vector based on the correlation matrix;
calculating a principal component fraction matrix from the structural characteristic vector; and
identifying at least one chromatin structural characteristic in the principal component fraction matrix.

2. The method for identifying the chromatin structural characteristic according to claim 1, wherein the Hi-C matrix is a raw-data Hi-C matrix.

3. The method for identifying the chromatin structural characteristic according to claim 1, wherein the Hi-C matrix is a normalized Hi-C matrix.

4. The method for identifying the chromatin structural characteristic according to claim 1, wherein the correlation process is at least one process selected from the group consisting of Pearson correlation, Spearman correlation, and cosine similarity.

5. The method for identifying the chromatin structural characteristic according to claim 1, wherein calculating the structural characteristic vector based on the correlation matrix includes at least one of calculating a quantile of the correlation matrix and characterizing similarity between a locus and at least one neighbor of the locus.

6. The method for identifying the chromatin structural characteristic according to claim 5, wherein calculating the structural characteristic vector based on the correlation matrix includes calculating the quantile of the correlation matrix.

7. The method for identifying the chromatin structural characteristic according to claim 6, wherein the correlation matrix is converted into a binary matrix, and
matrix elements greater than the quantile are converted to 1 or 0 and matrix elements less than the quantile are converted to the other of 1 or 0.

8. The method for identifying the chromatin structural characteristic according to claim 5, wherein calculating the structural characteristic vector based on the correlation matrix includes characterizing similarity between at least one locus and at least one neighbor of the locus.

9. The method for identifying the chromatin structural characteristic according to claim 8, wherein, for each locus, an average similarity between neighbor loci in a window is calculated,
a sub-matrix is generated from the correlation matrix based on a size of the window, and
the sub-matrix is averaged into the structural characteristic vector having a length equal to a number of chromatin bins.

10. The method for identifying the chromatin structural characteristic according to claim 1, wherein calculating the principal component fraction matrix from the structural characteristic vector includes:
splicing the structural characteristic vector into an input matrix with a defined shape so that each row of the input matrix is a structural eigenvector;
normalizing the input matrix; and
performing matrix decomposition and dimensionality reduction to obtain a coefficient matrix and the principal component fraction matrix.

11. The method for identifying the chromatin structural characteristic according to claim 10, wherein performing matrix decomposition and dimensionality reduction includes at least one selected from the group consisting of principal component analysis, non-negative matrix decomposition eigenvalue decomposition, and singular value decomposition algorithm.

12. The method for identifying the chromatin structural characteristic according to claim 1, wherein identifying the at least one chromatin structural characteristic in the principal component fraction matrix includes performing geometric visualization.

13. The method for identifying the chromatin structural characteristic according to claim 12, wherein the geometric visualization is a visualized cell type atlas.

14. A non-transitory computer readable medium storing a program for identifying a chromatin structural characteristic from a Hi-C matrix, the program causing a processor to execute:
performing a correlation process on the Hi-C matrix to calculate a correlation matrix;
calculating a structural characteristic vector based on the correlation matrix;
calculating a principal component fraction matrix from the structural characteristic vector; and
identifying at least one chromatin structural characteristic in the principal component fraction matrix.

15. A method for diagnosing a medical condition or disease, comprising:
identifying the chromatin structural characteristic according to the method of claim 1; and
relating the chromatin structural characteristic to a medical condition or disease.

16. The method for diagnosing a medical condition or disease according to claim 15, wherein the medical condition or disease is selected from the group consisting of cancer, cardiovascular disease, kidney disease, autoimmune disease, pulmonary disease, liver disease, lymphoid disease, bone marrow disease, bone disease, and blood disorder.

17. A method for treating a medical condition or disease, the method comprising:
identifying the chromatin structural characteristic according to the method of claim 1; and
administering a gene therapy vector to a subject in need thereof,
wherein the chromatin structural characteristic is indicative of a medical condition or disease.

18. The method for treating a medical condition or disease according to claim 17, wherein the gene therapy includes usage of transcription or translation production of at least one locus associated with the chromatin structural characteristic in target cells as a medical condition or disease target.

19. The method for treating a medical condition or disease according to claim 17, wherein the medical condition or disease is selected from the group consisting of cancer, cardiovascular disease, kidney disease, autoimmune disease, pulmonary disease, liver disease, lymphoid disease, bone marrow disease, bone disease, and blood disorder.

20. The method for treating a medical condition or disease according to claim 19, wherein the medical condition or disease is cancer.
